(19) United States
(12) Patent Application Publication
(10) Pub. No.: US 2015/0283334 A1
(43) Pub. Date: Oct. 8, 2015

(54) METHOD OF TUNING A VIBRATING MEDICAL DEVICE AND A CONNECTOR FOR THE SAME

(71) Applicant: VIBROVEIN PTY LTD, Armadale (AU)

(72) Inventors: John Alfred Marx, Southbank (AU); Nicholas Jon Ede, East Melbourne (AU); Toby James Hartley, Ferntree Gully (AU)

(21) Appl. No.: 14/437,308

(22) PCT Filed: Oct. 29, 2013
(86) PCT No.: PCT/AU2013/001253

§ 371 Date: Apr. 21, 2015

(30) Foreign Application Priority Data
Oct. 29, 2012 (AU) 2012904755
Sep. 6, 2013 (AU) 2013903427

(51) Int. Cl. A61M 5/34; A61M 5/42; A61B 17/32; A61M 39/10

(52) U.S. Cl. CPC A61M 5/345; A61M 39/10; A61M 5/422; A61B 17/320068; A61B 2017/00477

(57) ABSTRACT

A connector for use with a medical device is provided. The connector comprises a body portion having a first end and a second end. The body portion includes an internal channel extending along a length of the body portion providing fluid communication between the first end and the second end and a coupling connected to the body portion. The coupling comprises a first bracket arranged to receive a motor housing, wherein the first end is arranged to connect to an instrument body of the medical device and the second end is arranged to connect to an instrument tip of the medical device.

### FIELD OF THE INVENTION

Some embodiments relate to a device and method of performing medical procedures. More particularly, some embodiments relate to a connector for a device for causing vibration of a medical instrument having a cutting edge or a piercing edge/tip, and the performance of certain medical treatments using such a vibrating instrument. Furthermore, some embodiments relate to a control system dock and a method of calibrating a controller for tuning a vibration of a medical device.

### BACKGROUND

In this specification where a document, act or item of knowledge is referred to or discussed, this reference or discussion is not an admission that the document, act or item of knowledge or any combination thereof was at the priority date, publicly available, known to the public, part of common general knowledge; or known to be relevant to an attempt to solve any problem with which this specification is concerned.

While the following discussion focuses on a needle and syringe, some embodiments have application to a wide range of medical instruments having a cutting edge.

The use of a syringe and needle to introduce fluids into the body or to draw fluid (ie. blood) for medical purposes is known. Although patient discomfort and pain associated with injections can be reduced in some circumstances by the use of small gauge needles and good technique, pain cannot always be adequately eliminated particularly when multiple injections are required. Many patients suffer from phobias about receiving injections and may avoid medical treatment due to fear of injections.

Modern manufacturing techniques are able to produce extremely sharp cutting edges on the tips of needles and other medical instruments such as scalpels, however these sharpened surfaces still require a force to be applied to penetrate body tissues. Many of those tissues (such as skin or the walls of a vein) still require a reasonable force to penetrate the tough connective tissue or tissue layers which can cause the tissue to be stretched or deformed from its usual shape. For example, if an injection force is applied to delicate skin in certain areas of the face (such as the lips or around the eyes), the skin will be deformed by the pressure of the needle required to penetrate that skin, particularly with more mature skin which may have lost some of its elasticity. Similarly, during the treatment of diseased or abnormal veins (such as varicose or spider veins), the vein walls may be unduly deformed by the force required for the needle cutting tip to penetrate the wall. It is this deformation which leads to the patient feeling pain during treatment, despite the sharp cutting edges. The pain is proportional to the amount of skin and tissue deformation caused by the passage of the needle through the skin and underlying tissue. The amount of skin and tissue deformation will be determined by the following needle parameters:
penetration resistance; and
stiction (which is explained below).
   This is because the cutting edge is only relatively sharp and is, in fact, blunt at the microscopic level.

The deformation is a result of “static friction” or “stiction”. Stiction refers to the phenomenon when the shaft of the needle is in frictional contact with the tissue through which the needle tip has penetrated. For example, tissues that are effectively visco-elastic (such as the skin or a vein wall) will generally deform until the force applied to the needle tip is sufficient for tip penetration to occur. In addition, as the needle advances through a tissue, static friction between the exterior walls of the needle shaft and the tissue may cause the needle to drag, and thus deform the surrounding tissue in the direction of the advance.

In several cosmetic restorative procedures, for example sclerotherapy, wrinkle reduction or administration of dermal fillers, it is also highly desirable to reduce the deformation of the subject area caused by penetration resistance and stiction for the following reasons.
although it is possible to use ultrasound imaging to guide a fine gauge needle for such procedures, the vein wall still may become distorted and the accuracy of needle placement may frequently be compromised;
inaccurate placement of the needle may cause trauma to the vein wall, in particular, stripping of the intima layer. The result of stripping of the intima layer is a failure of the endovenous laser fibre to successfully enter into the veins lumen. The trauma caused by the stripping of the lumen usually means it will not be possible to successfully enter the laser fibre into the veins lumen, and the whole procedure will have to be aborted; and
it can become difficult to see the wrinkle and ensure that the injection is given at the most appropriate location.

In addition, some of the substances to be administered in facial injections are relatively viscous and may require a large gauge needle for administration, however this would create a greater penetration resistance and stiction problem and the associated pain would not be acceptable. Indeed, even with fine gauge needles, practitioners regularly administer a local anaesthetic prior to giving cosmetic treatments for patient comfort.

There have been a number of attempts to address these difficulties. One example of such an attempt is the vibrating medical device disclosed in international patent application no WO 2008/086560. However, WO 2008/086560 discloses use of any frequency in the range from 50 to 20,000 Hz, more preferably 100 to 10,000 Hz and even more preferably between 200 and 500 Hz. The embodiment described in WO 2008/086560 has a motor with a maximum frequency of 420 Hz. Other examples include the vibrating medical devices in US 2004/0243136, PL165506, US 2008/0294087, US2007/0135827, EP1693027, US 2005/0234484, US 2008/0319446 and WO 2003/024513. Many of these documents do not discuss the frequency at which the device vibrates. None of these attempts has fully addressed the above difficulties. Accordingly, there is still a need for further improvement in addressing the difficulties discussed above.

### SUMMARY

Some embodiments relate to a device and method of performing medical procedures. More particularly, some embodiments relate to a connector for a device for causing vibration of a medical instrument having a cutting edge or a piercing edgetip, and the performance of certain medical treatments using such a vibrating instrument. Furthermore, some embodiments relate to a control system dock and a method of calibrating a controller for tuning a vibration of a medical device.

Some embodiments relate to a connector for use with a medical device, the connector comprising a body portion having a first end and a second end, the body portion including an internal channel extending along a length of the body portion and in fluid communication with or allowing fluid communication between the first end and the second end and a coupling connected to the body portion, the coupling comprising a first bracket arranged to receive a motor housing, wherein the first end is arranged to connect to an instrument body of the medical device and the second end is arranged to connect to a instrument tip of the medical device. In one embodiment, the coupling may be rigidly attached to the body portion.

In one embodiment, the first bracket may comprise first and second arms, each arm extending outwardly from the body portion and substantially toward one another to define an enclosure for receiving the motor housing.

In one embodiment, the coupling may comprise a second bracket arranged to engage with the body portion.

In one embodiment, the coupling may comprise a spine portion, wherein the first bracket extends from the spine in a first direction and the second bracket extends from the spine in a second other direction.

In one embodiment, the second bracket may comprise first and second arms, each arm extending outwardly from the spine of the coupling and substantially toward one another to define an enclosure for receiving the body portion.

In one embodiment, a plurality of detents may be disposed on a surface of the body portion and may be arranged to engage with an inner surface of the second bracket to thereby allow the coupling to be rotated incrementally about the body portion. In some embodiments, the plurality of detents may extend at least partially around a circumference of the body portion to thereby provide a plurality of discrete increments by which the coupling may be rotated incrementally about the body portion.

In some embodiments, a fitting may be provided at the first end of the body portion and may be arranged to cooperate with a corresponding fitting provided on the instrument body. In some embodiments, a fitting may be provided at the second end of the body portion and may be arranged to cooperate with a corresponding fitting provided on the instrument tip. The fitting may comprise a Luer taper. The Luer taper may comprise a male or female portion of a Luer lock or slip tip, such as the Beckton Dickenson Luer-Lok® or Luer-Slip®.

Some embodiments relate to a medical device comprising an instrument body, an instrument tip including a needle and a connector as set out above, wherein the connector provides fluid communication between a chamber of the instrument body and the needle.

According to some embodiments, there is provided a device for use with a medical instrument having a body and a cutting edge or piercing tip extending from the body, the device comprising a vibrator for causing the cutting edge or piercing tip of said instrument to vibrate, wherein the vibrator vibrates at the optimum penetration frequency of the cutting edge or piercing tip.

According to some embodiments, there is provided a control system dock comprising a base unit including a processor, a controller cradle arranged to receive a controller, a fixture arranged to receive a medical device and a sensor, wherein the processor is operable to receive signals from the sensor relating to a measure of vibration of an instrument tip of the medical device and to transmit signals to the controller to program the controller to transmit power to a vibrating motor connected to the medical device. In some embodiments, the processor may be operable to program the controller to transmit an amount of power to the vibrating motor to cause the vibrating motor to vibrate at a particular frequency. The processor may communicate with the controller via WiFi, Bluetooth, a physical connector or via any suitable communications network.

In some embodiments, the controller cradle includes electrical terminals arranged to electrically connect to the controller and arranged to transmit power from the control system dock to the controller. In some embodiments, the sensor comprises a frequency sensor. In some embodiments, the sensor comprises a motion sensor. In some embodiments, the sensor comprises a frequency and motion sensor.

In some embodiments, the processor is arranged to receive user inputs via user actuatable controls provided on the control system dock. In some embodiments, the processor is arranged to provide outputs via a display provided on the control system dock.

In some embodiments, the processor may be operable to receive a feedback signal from the controller in response to an adjustment of the power being transmitted by the controller. The information derived from the feedback signal may be employed for further operations. For example, the information may be stored in a memory accessible to the processor and/or may be employed for future calibrations of the controller and/or tuning of the medical device.

In some embodiments, the control system dock may comprise a controller provided in the controller cradle and a medical device provided on the fixture, wherein the medical device may comprise a connector including a vibrating motor. For example, the medical device may include a connector of the type described above. In some embodiments, the controller may comprise a user interface to allow for adjustment of the power to be transmitted to the vibrating motor. In some embodiments, the controller may be a battery operated hand-held controller. The controller may be connected to the vibrating device by a cable. In some embodiments, the base unit may be connected or connectable to a mains power supply.

According to some embodiments, there is provided a method of calibrating a controller for use with a medical device, the method comprising transmitting power from a controller to a vibrating device connected to a medical device, receiving a measurement value indicative of a vibration of a tip of the medical device, and subject to determining that the measurement value does not fall within an acceptable range, transmitting from a processor to the controller, a signal to adjust an amount of power being transmitted to the vibrating device.

In some embodiments, the method may further comprise, at the controller, adjusting the amount of power being transmitted to the vibrating device. The adjusting may comprise increasing or decreasing the amount of power being transmitted to the vibrating device.

In some embodiments, determining that the measurement value does not fall within an acceptable range may comprise the processor comparing the measurement value with a threshold range or value stored in a memory. In some embodiments, determining that the measurement value does not fall within an acceptable range may comprise providing a user with an indication of the measurement and receiving a user input indicative of whether or not the measurement falls within an acceptable range. In some embodiments, transmitting the signal to adjust an amount of power being transmitted to the vibrating device is performed in response to a user input.

In some embodiments, receiving a measurement value indicative of a vibration of a tip of the medical device comprises sensing at least one of an oscillation frequency of the tip and an oscillation amplitude of the tip.

In some embodiments, the method further comprises receiving a feedback signal from the controller in response to an adjustment of the power being transmitted by the controller. The adjustment of the power being transmitted by the controller may be in response to a user input received via a user interface provided on the controller. The information derived from the feedback signal may be employed for further operations. for example, for future calibrations of the controller and/or tuning of the medical device.

Some embodiments relate to a vibration of a medical instrument during medical procedures to reduce some of the difficulties discussed above. It has surprisingly been found that the use of vibrations at a particular frequency provides advantages with respect to the efficiency of the medical instrument. The optimum penetration frequency is the frequency at which the cutting edge or piercing tip will vibrate at its optimum rate so that the penetration resistance (i.e. the pressure required for penetration) of the cutting edge or piercing tip and stiction of the medical instrument is effectively or substantially minimised, and at which it is still possible to use the medical instrument for the intended procedure (for example, the vibration/oscillation does not interfere with the ability to be accurate in the placement of the medical instrument). It has surprisingly been found that the optimum penetration frequency is one where there is some oscillation of the cutting edge or piercing tip when the cutting edge or piercing tip is not dampened (i.e., in mid-air), although this will not always be visible, rather than a frequency at which the cutting edge or piercing tip achieves a stationary node. The optimum penetration frequency may be a balance between the ideal frequency to minimise the penetration resistance and stiction and the amount of oscillation caused by the vibration with respect to being able to properly use the medical instrument.

The optimum penetration frequency for a particular cutting edge or piercing tip will depend on the cutting edge or piercing tip itself, the body of the medical instrument and the vibrator. The vibrator can be any suitable device for providing vibration or pulses to the medical instrument, such as piezoelectric devices and standard electric motors (as discussed in more detail below). The optimum penetration frequency for a cutting edge or piercing tip could therefore be in the sonic or ultrasonic frequencies depending on the vibrator.

An example of a cutting edge or piercing tip is the needle of a syringe. Each needle size will have a different optimum penetration frequency with different syringes. In one preferred embodiment, where a motor is used as a vibrator, the vibrator causes vibration in a needle connected to a syringe and the optimum penetration frequency, when measured using a Testometric M250-2.5CT tensile testing instrument which utilized a load cell on a linearly actuated arm holding a 5 ml Terumo syringe to determine the resulting penetration of tattoo practice skins as a function of force (see Example 1 for full details), was:
570 to 670 Hz for a 25G×1.5 inch needle;
570 to 670 for a 200×1.5 inch needle;
350 to 500 Hz for a 30G×0.5 inch needle; and
450 to 570 Hz for a 18G×1.5 inch needle.

In some embodiments, there is provided a method of reducing the resistance to penetration of a body tissue by a cutting edge or piercing tip on a medical instrument, the penetration of said body tissue occurring along an axis, the method comprising:
(a) tuning a vibrator to vibrate so as to cause said cutting edge or piercing tip to vibrate at the optimum penetration frequency of the cutting edge; and
(b) using said vibrating cutting edge or piercing tip when penetrating the body tissue.

Depending on the medical instrument and vibrator used, the tuning step (a) could be achieved by any suitable method, including sight (e.g. looking for the oscillation of the cutting edge or piercing tip in mid-air), sound (listening for the harmonics), or an oscilloscope. With the longer needles used in syringes (e.g. 1.5 inch), it is possible to visually tune the needle to the optimum penetration frequency because the needle when free to move in air will oscillate in a V-shaped oscillation (see FIG. 6 for an example) and the frequency of vibration can then be adjusted to achieve a level of oscillation which allows for proper use of the medical instrument. In contrast, at a non-optimum frequency as per the prior art, a stationary node is obtained in the needle (see FIG. 7).

In some embodiments, the cutting edge is remote from the body and is moveable along an axis, and the vibrator causes the cutting edge or piercing tip to vibrate in a motion substantially transverse to said axis. For example, when the medical instrument is a needle and syringe, the axis of movement of the needle will be along the length of the needle, i.e. the needle would be inserted into or through a body tissue along its length. The needle would be caused to vibrate at 90° to this axis of movement.

The device, when attached to a medical instrument, may cause the cutting edge or piercing tip to vibrate in a circular motion substantially transverse to the axis, although other types of motion, such as elliptical or linear motion transverse to the axis, may be acceptable to create the desired result depending the procedure being administered.

In some embodiments, the device further comprises an electronic means for analyzing the optimum penetration frequency of the cutting edge or piercing tip. For example, the resistance to cutting of tissue (e.g. pressure) may be measured and correlated to the frequency being applied by the vibrator to the cutting edge or piercing tip. The frequency being applied by the vibrator could then be adjusted via the use of a feedback loop to the vibrator, so that the optimum penetration frequency of vibration can be automatically applied to the cutting edge or piercing tip of the medical instrument.

The device may comprise a controller for selectively causing said vibrator to vibrate at the optimum penetration frequency. The controller for selectively causing said vibrator to vibrate at a selected frequency may include:
a first sensor for sensing the ease by which the cutting edge or piercing tip penetrates body tissue,
a second sensor for sensing the frequency of the vibration of the vibrator,
a correlation device to correlate the measurements sensed by the first and second sensors and provide a feedback signal; and
an adjuster to select the frequency for the vibration of the vibrator in response to the feedback signal.

Some embodiments extend to a medical instrument which comprises a device of the above described kind.

In some embodiments, there is provided a method of reducing the static friction between a cutting edge or piercing tip and a body tissue when said cutting edge or piercing tip is forced against said body tissue along an axis, the method comprising causing said cutting edge to vibrate at the optimum penetration frequency of the cutting edge or piercing tip.

In some embodiments, there is provided a method of performing sclerotherapy comprising connecting to the body of a syringe bearing a needle a sterilisable battery powered vibrator and introducing the needle into a vein while said vibrator vibrates said needle at the optimum penetration frequency of the needle.

In some embodiments, there is provided a method of injecting a substance into the face of a patient with a hypodermic needle and syringe comprising mounting a vibrator on the barrel of said syringe, causing said cutting edge or piercing tip to vibrate at the optimum penetration frequency of the cutting edge or piercing tip and then injecting the substance. In addition to reducing the effects of penetration resistance and stiction between the needle and the skin of the patient, it has been observed that this method has an advantageous benefit in reducing pain to the injection site.

In some embodiments, the vibrator may take a variety of forms. Suitable devices include piezoelectric crystal devices (such as a transducer) and standard electric motors. Other options include hydraulic, pneumatic or mechanical servosystems and a permanent magnetic micro-vibration motor.

Pulses or vibrations could be generated in a unit which is attached to the medical instrument or the pulses or vibrations could be generated remotely from the instrument and transferred to the instrument by any suitable arrangement, provided that the resultant vibration created would be at the optimum penetration frequency of the medical instrument.

The vibrator may be powered through a controller where the frequency of vibration may be varied by the operator to a selected level.

In one embodiment, the vibrator is a small, unobtrusive motor. The motor may include a shaft bearing on eccentric weight, and the axis of the shaft is substantially parallel to the longitudinal axis along of the body when the device is attached to the body.

In one embodiment, the motor is powered remotely from the medical instrument when the device is attached to the instrument. Alternatively, a capacitor could store electrical energy to drive a suitable vibrating motor.

In another embodiment, the device may be an internally powered device including a motor and battery located within a housing which is contained within resilient means made from a waterproof elastomeric material such as a rubber, for example, a latex or silicone rubber which is readily sterilisable. Suitable resilient materials are well known in the medical arts. The housing and resilient means may be formed as a single integral component. The resilient means may be extensible by more than 100%, or by more than 300%, of its resting length without breaking. The resilient means may be one or more rings extending from said housing which can be stretched around the body of the medical instrument.

In this embodiment, the battery operated vibrating motor is a lightweight (between about 3 to 10 grams) electric motor having an eccentrically weighted shaft which transmits vibration to the housing. The motor may be contained within a shell, the whole assembly of which can be sterilized. The entire device may be of a sufficiently simple design and has few parts such that it can be mass produced cheaply, packaged in an individual sterile package and is suitable for a single use only. Thus, the device may be sterilisable during manufacture, and can be disposed of after a single use. The battery may be a small, single cell, of a size similar to a watch battery, and which is capable of powering the motor at the desired rate of revolutions for at least 10 minutes.

Where battery power is employed, the battery may be provided within the housing which contains the vibrating motor or it can be remote from the housing. For example, a battery pack might be provided remote from the housing and a suitable electric connection made through the housing with the vibrating motor. The battery pack could be rechargeable and therefore reusable. It could also be positioned sufficiently remote from the medical instrument so that it could be placed on an equipment table. In one embodiment, the battery pack may be strapped to an arm of a practitioner or patient. By removing the battery from attachment to the medical instrument, or by providing an external source of mains power to energise the device, the bulk and weight of the attachment can be reduced.

Some embodiments further include a vibrating motor which is not connected to the medical instrument with which it is employed, but which is brought into contact with that instrument when the instrument is to be vibrated. In one embodiment of this kind, a vibrating motor can be applied to one or more fingers of the practitioner who is using the medical instrument, so that when the instrument is held by the practitioner, the vibrations transfer to the medical instrument and the vibrating effect is achieved.

In some embodiments, the device is highly versatile and may be applied to a wide range of medical instruments, thus its potential applications should be considered broadly.

The medical instrument may be of any suitable kind used in medicine, where overcoming penetration resistance and/or stiction is advantageous, or the other benefits referred to below are desirable. For example, medical instruments used in procedures where the improved mixing of fluids either outside or within the body is desirable.

In the case of a hypodermic needle, the cutting edge or piercing tip is the piercing and cutting tip of the needle. Depending on the size of the needle, the device may be attached to either the barrel of the syringe to which the needle is mounted or, if a sufficiently large needle is being used, the needle itself.

Where the medical instrument is a scalpel, the device may be mounted on the handle of the scalpel.

The cutting edge or piercing tip on the medical instrument may be any cutting edge or piercing tip used in medicine, for example a scalpel blade, or a penetrating tip of a hypodermic needle, or a lancet. The cutting edge or piercing tip may be a bevelled edge of a needle.

More preferably, the medical instrument may be a hypodermic needle and the cutting edge or piercing tip may be the tip of the needle.

In some embodiments, the device comprises a medical instrument which can be used in endovenous laser therapy. In such therapy, a small laser fiber is inserted through a needle into a damaged vein and is guided within the vein by a U-shaped guide wire. The heat generated by pulses of laser light delivered into the vein causes the vein walls to collapse and seal shut. In this procedure, the U-shaped guide wire sometimes sticks into the vein wall as it is fed into the vein causing difficulty in feeding the guide wire to the correct position. Some embodiments may alleviate this difficulty by causing the guide wire to vibrate and thus to reduce or eliminate sticking of the guide wire into the vein wall.

Where the vibrator is connected to the medical instrument, the connection of the vibrator to the medical instrument must be accomplished in a manner which minimises any motion of the vibrator relative to the medical instrument and enables the vibrations to be transmitted to the medical instrument, but at the same time does not significantly interfere with the operation of the medical instrument. For example, where the medical instrument is a syringe, it is important that the attachment to the syringe does not compress and distort the shape of the plastic syringe to such an extent that it significantly compromises the movement of the plunger inside the syringe.

One coupling device for attaching a vibrator to a medical instrument, such as a syringe, is a resilient spring or clip wherein the diameter of the spring winding or the clip is slightly smaller than the diameter of the medical instrument, but the winding/clip is not so tight as to compress or distort the medical instrument. Such a coupling device can be made of any suitable material, for example, any suitable resilient plastic such as perspex. The resilient plastic may be either heat moulded from a flat piece of plastic or else extruded through a mould into a long piece of plastic which is then cut into appropriately sized segments. Where the medical instrument needs to be viewed, for example the measurements on a syringe, then preferably the coupling device is made from a transparent material.

One example of such a coupling device is a spring having a first winding in a clockwise direction and a second winding in an anti-clockwise direction. Such a spring could have either a ‘figure of 8’ shape or a ‘B’ shape. The vibrator will resiliently fit into one winding, and the medical instrument will resiliently fit into the other winding. For example, the winding for the vibrator may have a diameter of 0.8 mm whereas the vibrator housing may have a housing of 1.0 cm. This coupling device also has the advantage of being able to be rotated about the medical device so as to enable the vibrator to be positioned so as to cause the cutting edge or piercing tip to vibrate in a motion substantially transverse to the axis of movement.

### DRAWINGS

Various embodiments/aspects will now be described with reference to the following drawings in which:

FIG. 1 is a graph illustrating the results from Example 1 for the 20G×1.5′ needle;

FIG. 2 is a graph illustrating the results from Example 1 for the 25G×1.5″ needle;

FIG. 3 is a graph illustrating the results from Example 1 for the 30G×0.5″ needle;

FIG. 4 is a graph illustrating the results from Example 1 for the 18G×1.5″ needle;

FIG. 5a is a top view of a coupling device according to some embodiments;

FIG. 5b is a perspective view of the coupling device of FIG. 5a

FIG. 6 is a side view of a 1.5″ needle when stationary and when oscillating at the optimum penetration frequency according to some embodiments;

FIG. 7 is a side view of a 1.5″ needle with a stationary node using a frequency as per the prior art;

FIG. 8a is a perspective view of a medical device comprising a connector interconnecting an instrument body and an instrument tip, according to one embodiment;

FIG. 8b is an exploded view of the medical device of FIG. 8a;

FIG. 9a is a top view of the medical device of FIGS. 8a and 8b;

FIG. 9b is a cross sectional view taken along the line A-A of FIG. 9a;

FIG. 10a is a perspective view of a medical device comprising a connector interconnecting an instrument body and an instrument tip, according to one embodiment;

FIG. 10b is an exploded view of the medical device of FIG. 10a;

FIG. 11a is a top view of the medical device of FIGS. 10a and 10b;

FIG. 11b is a cross sectional view of the medical device taken along the line A-A of FIG. 11a;

FIG. 11c is a cross sectional view of the medical device taken along the line B-B of FIG. 11a;

FIG. 12a is a perspective view of the connector of the medical device of FIG. 10a comprising a male fitting of a Luer-Lok®;

FIG. 12b is a perspective view of the connector of the medical device of FIG. 10a comprising a male fitting of a Luer-Slip®;

FIG. 13a is a perspective view of a control system comprising a controller arranged to electrically connect to a vibrating motor provided within a motor housing, according to some embodiments;

FIG. 13b is a front view of the control system of FIG. 13a;

FIG. 14a is a perspective view of a control system dock arranged to receive the control system of FIGS. 13a and 13b;

FIG. 14b is a perspective view of the control system dock of FIG. 14a and the control system of FIGS. 13a and 13b; and

FIG. 15 is a flow diagram of a method for calibrating controller for use with a medical device of FIG. 8a or FIG. 10a.

### DETAILED DESCRIPTION

Various embodiments/aspects will now be described in more detail. It is to be understood that the drawings and the following description relate to preferred embodiments only and are not intended to limit the scope of the invention.

The general principles in developing a suitable vibrator disclosed in international patent application no WO 2008/086560 are hereby incorporated by reference in their entirety. The vibrator will need to be able to cause the cutting edge or piercing tip to vibrate at the optimum penetration frequency of the cutting edge or piercing tip.

The gentle, unforced, substantially frictionless needle movement observed in some embodiments creates significantly less pain and damage to surrounding tissues, and enables a practitioner to target the desired site, such as a vein, with improved skill and accuracy.

The benefits which have been discovered include:
penetration of patient skin and other subsequent organs is less painful to the patient and causes less damage (deformation) to surrounding tissues;
penetration of patient skin and subsequent organs is more accurate. The increased accuracy may result from the need for less plunger pressure by the practitioner;
the cutting edge or piercing tip of a medical instrument tends to have greater purchase, so that body tissues such as veins (which tend to shift under the pressure of a non-vibrating needle cutting edge or piercing tip for example) tend to remain in position rather than moving. This causes the rate of successful penetration of such body parts, the so-called “strike rate”, to be much higher, resulting in greater patient confidence, shorter procedure time and reduced body trauma; and
reduced needle failure and reduced blunting of needles during procedures where multiple needle insertions are required, such as in cosmetic procedures like injection of dermal fillers, or in sclerotherapy. The amount of needle blunting may correlate to the amount of pain and trauma inflicted on a patient. The reduction in needle blunting observed may then be a further indicator of reduced patient pain and trauma. The reduced blunting also lengthens the life of the needle so that it does not need to be replaced as often.

Accuracy has also been observed to increase in reticular vein strike rates when using a device of some embodiments. Reticular veins, particularly around the ankle and shin regions, are notoriously difficult to sclerose and therapists are often forced to abandon attempts to sclerose these veins after having created significant trauma to surrounding tissues. These veins are very mobile, with a significant amount of inherent movement and are therefore difficult to stabilize. If the practitioner misses the vein on the first attempt a haematoma is likely to form, with an underlying vein spasm. In some embodiments, such veins are much easier to sclerose, and even if the practitioner misses the vein initially, only minimal damage is likely to have occurred to surrounding tissues and a second attempt may then be made.

While not wishing to be bound by theory, the above benefits are considered to result from a reduction in the penetration resistance and stiction load experienced in some medical instruments, such as hypodermic syringes. Thus, penetration occurs more readily and without, or with significantly reduced, deformation of the body tissue. Moreover, because stiction loads are reduced, instruments such as syringes or scalpels tend to pass through the body tissue more easily and so with reduced body tissue deformation and significantly less blunting. Accordingly, the number of needles required to be used in, for example, a sclerotherapy session, may be significantly reduced.

During experimental trials, the applicant has observed a marked increase in the accuracy of placement when a device of some embodiments has been used during sclerotherapy treatments, of the needle tip using ultrasound guidance, and a lessening of distortion to the subject vein wall when the needle tip is being passed therethrough. The applicant has further found that penetration resistance and stiction between facial skin and needle during penetration is reduced, as is the pain encountered by patients during such procedures. The apparent viscosity of thick injectables used in such procedures is also reduced, thus improving their administration.

Various embodiments/aspects will now be described with reference to the following non-limiting example:

In this example, the variation in the penetration force required to penetrate skin with a vibrating needle of a syringe at different frequencies was investigated.

Testing was conducted on the Testometric M250-2.5CT tensile testing instrument which utilizes a load cell on a linearly actuated arm holding a 5 ml Terumo syringe. The resulting penetration of the test skin by the needle was determined as a function of force.

The experiment used tattoo practice skins from Funtopia (a business in Queensland, Australia). This skin caused substantial blunting of the needles so the test was conducted using a new needle for each penetration. There were also some difficulties experienced because the thickness of the material was not consistent. In real life, skin is not always the same thickness as it varies for different people, between males and females, and for different body parts.

The motor used was a 13 mm electrically commutated motor (part no 368851) controlled with a DECS 50/5 amplifier (part no 343523) purchased from Maxon Motor Australia. The frequency was changed by manipulating the potentiometer on the motor driver which had settings 1 to 11 (however settings 1 and 2 failed to rotate the motor shaft). The motor drive was connected to an oscilloscope to enable measurement of the input frequency, that is, the frequency of the motor. This will be different to the frequency of the needle as there will be some losses. The following table sets out the measurements obtained for the motor settings.

| Maxon motor setting | Motor frequency (Hz) |
|---|---|
| 3 | 110 |
| 4 | 180 |
| 5 | 255 |
| 6 | 326 |
| 7 | 420 |
| 8 | 480 |
| 9 | 570 |
| 10 | 635 |
| 11 | 670 |

The needles used for the testing were 20G×1.5′, 25G×1.5″, 30G×0.5″ and 18G×1.5″. For the single penetration tests, on penetration was performed for each motor frequency setting (including the static needle).

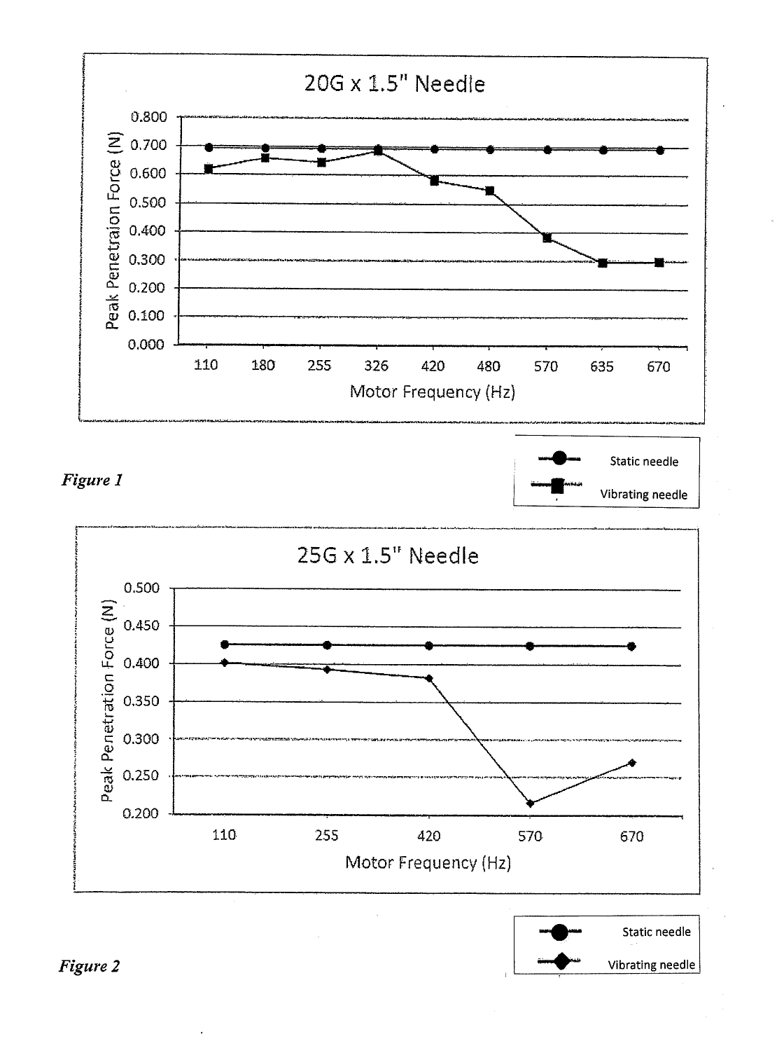

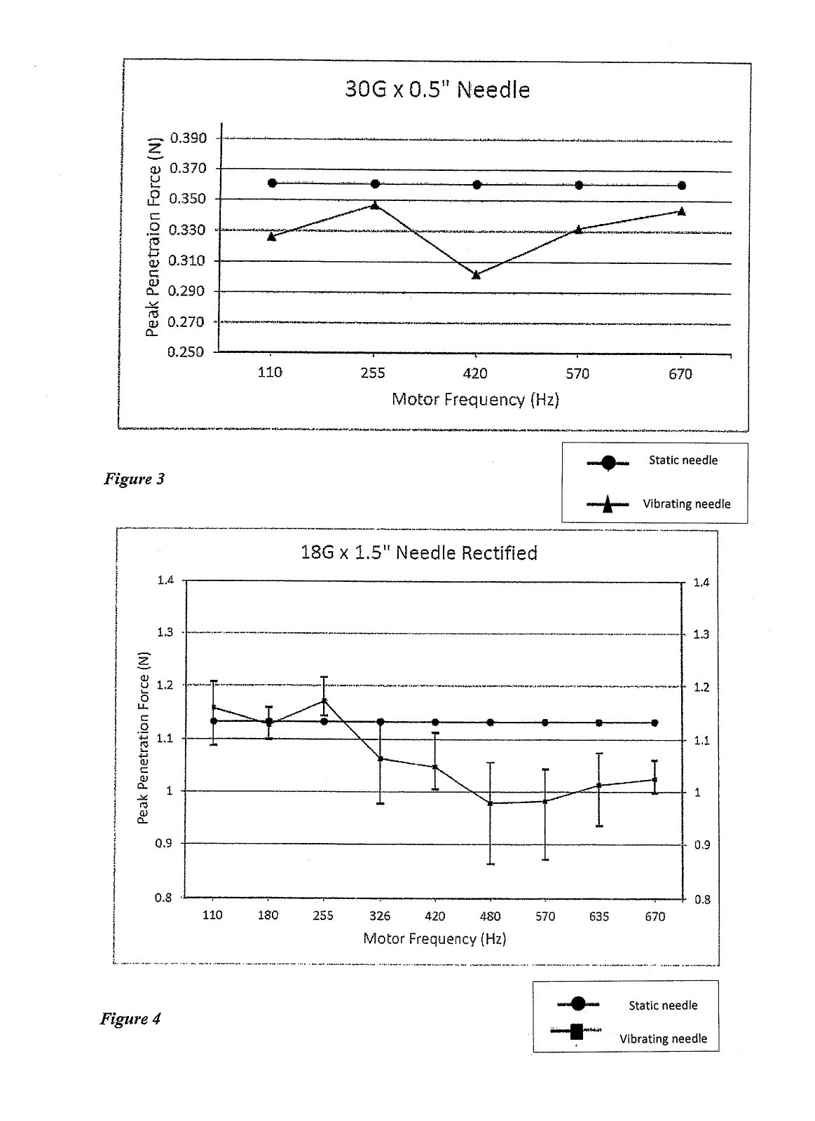

The results obtained from single penetration of the 20G×1.5′, 25G×1.5″ and 30G×0.5″ needles are illustrated in FIGS. 1 to 3, respectively. The peak penetration force for each frequency setting has been plotted with the squares trace. The circles trace represents the static needle.

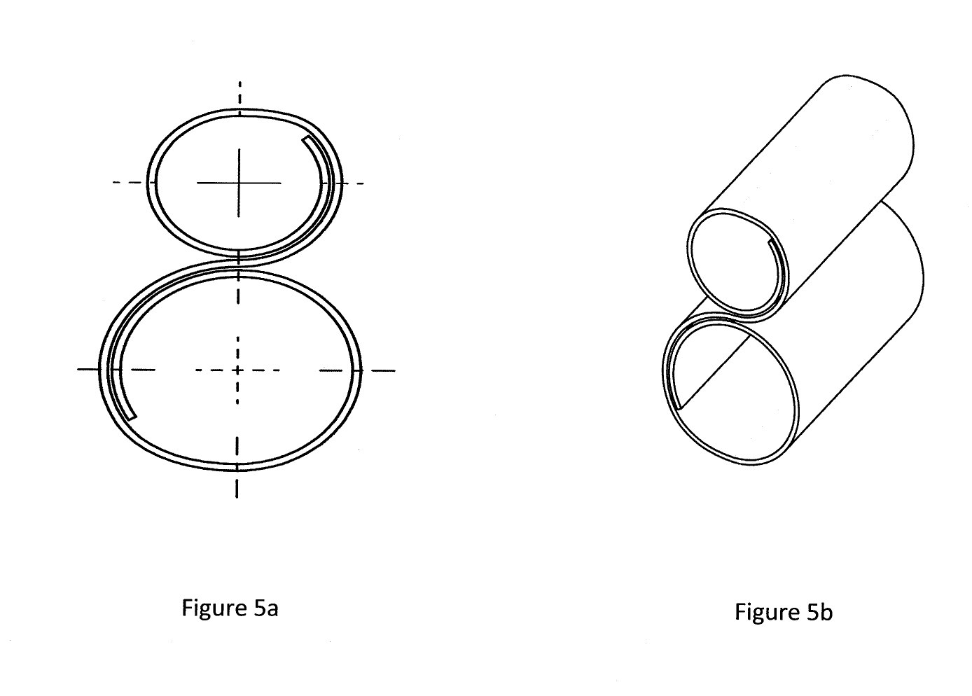

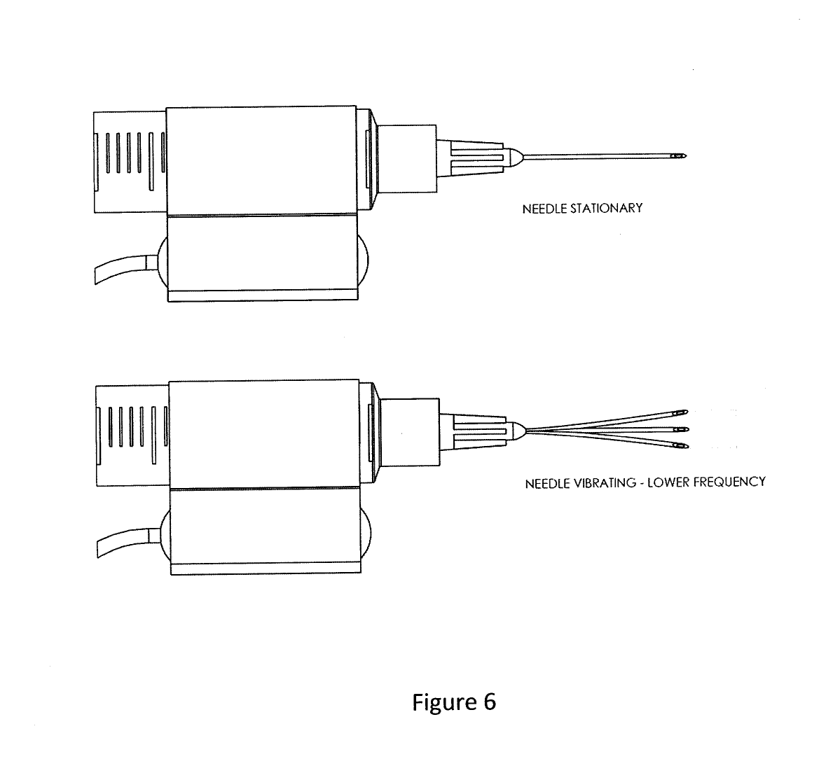

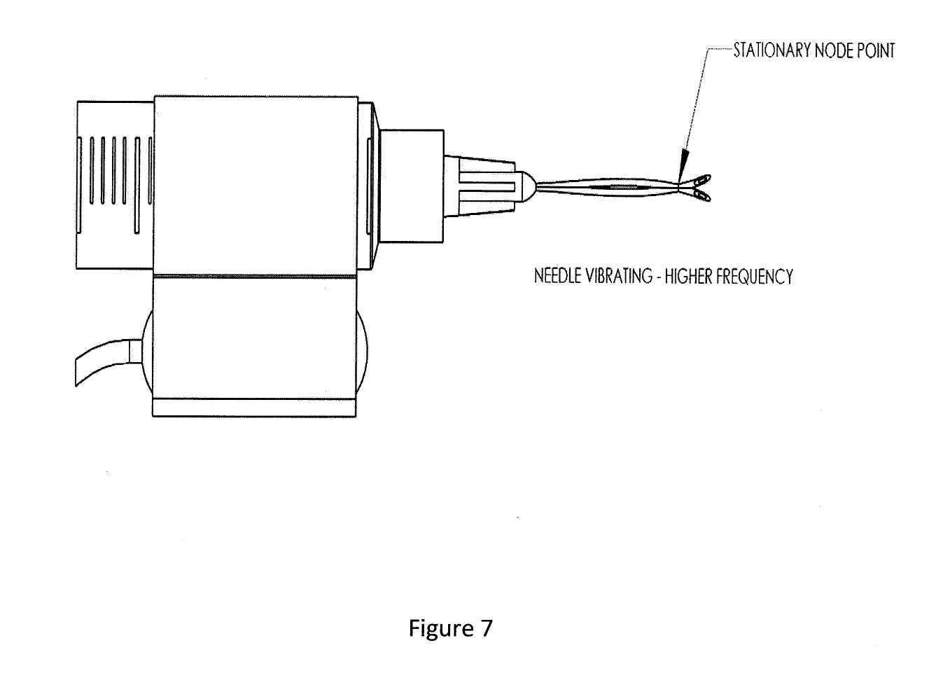

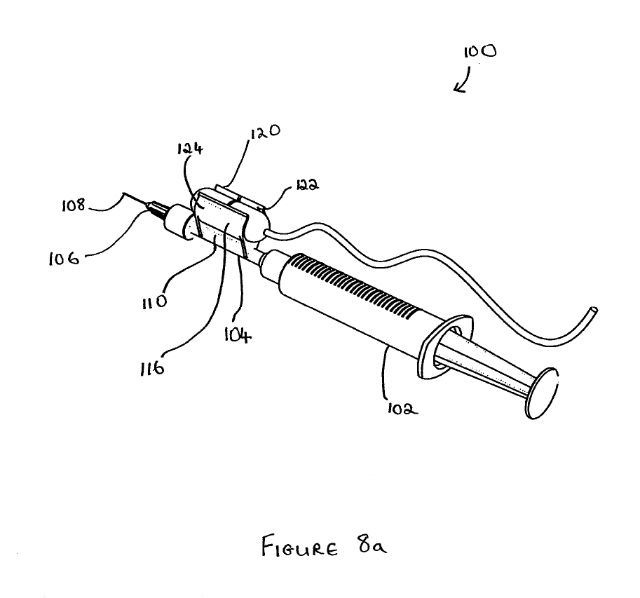

The results obtained from penetration of the 18G×1.5″ (n=6) are illustrated in FIG. 4. The results have been corrected to allow for the variable thickness of the tattoo practice skin.

These results show that whilst vibration does reduce the peak penetration force of a needle into skin, there is an ideal frequency where the peak penetration force of the needle is at its lowest (the optimum penetration frequency). The use of frequencies above or below this ideal frequency led to greater peak penetration forces being required. The results obtained for the 20G needle demonstrate that the use of the ideal frequency (at about 635 Hz) can lead to a reduction of almost 60% in peak penetration force (50% for 25G and 16% for 30G).

The increase in needle gauge from 20G to 18G increased the static penetration force dramatically (over 1.1 N of force). The use of vibration reduced the peak penetration force by 24% at 480 Hz.

FIGS. 5a and 5b are photographs of a coupling device according to a further aspect. The coupling device is made from a piece of transparent perspex which has been heat moulded into a ‘figure of 8’ shape so that the coupling device comprises a spring having a first winding in a clockwise direction and a second winding in an anti-clockwise direction.

The windings have a diameter of 0.8 mm which is slightly smaller than the diameter of the vibrator housing of 1.0 cm. This ensures that the vibrator is held snugly and does not move relative to the body of the medical instrument. At the same time, the resilient properties of the perspex are such that the other winding exerts just enough pressure on the body of the medical instrument, namely a syringe, but the winding is not so tight as to compress or distort the shape of the plastic syringe to such an extent that it compromises the movement of the plunger inside the syringe.

This coupling device also has the advantage of being able to be rotated about the medical device so as to enable the vibrator to be positioned so as to cause the cutting edge or piercing tip to vibrate in a motion substantially transverse to the axis of movement.

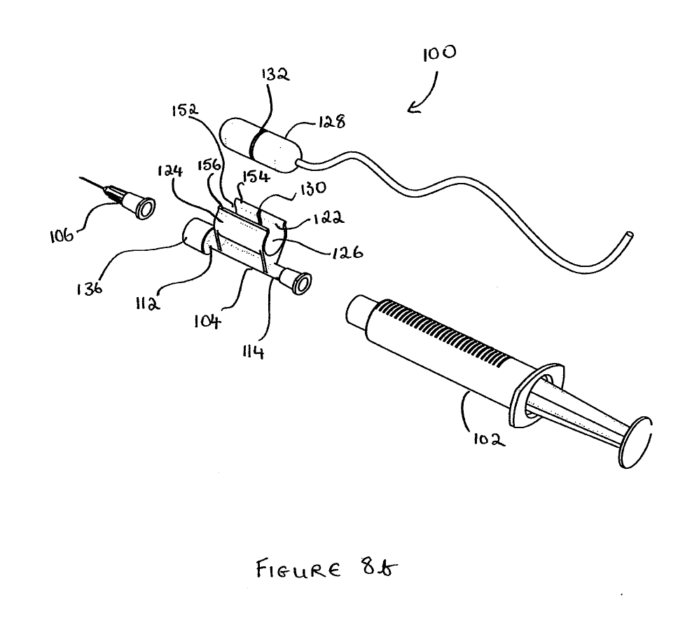

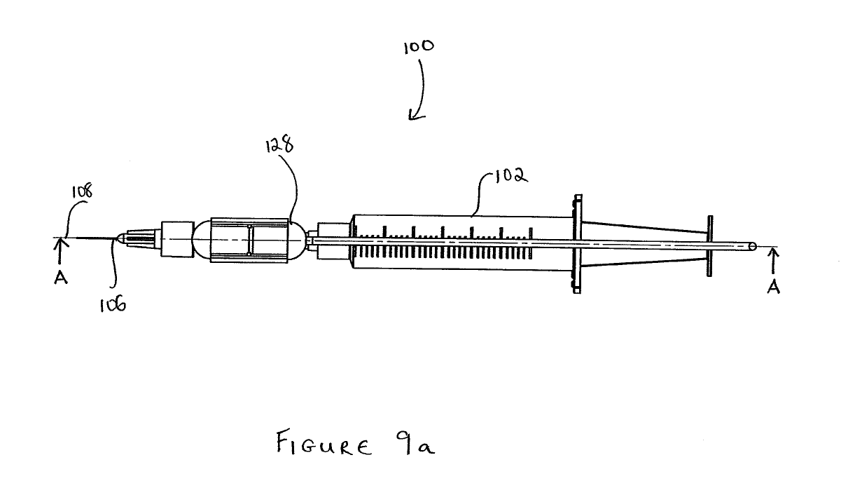

Referring now to FIGS. 8a and 8b, there is depicted a medical device 100 comprising a medical instrument body 102, such as a syringe, a connector 104, and a medical instrument tip 106, arranged to receive a needle 108, according to one embodiment.

The connector 104 may comprise a body portion 110 having a first and second end portion, 112, and 114, respectively. In one embodiment, the body portion 110 is a longitudinal body portion and may be substantially cylindrical in shape. However, the body portion may be shaped in any suitable manner, for example, such as a rectangular or hexagonal prism.

The connector 104 may further comprise a coupling 116. The coupling 116 may be securely attached to the body portion 110 to prevent or at least mitigate the coupling 116 from moving independently of the body portion 110. In one embodiment, the coupling 116 may be rigidly fixed to the body portion 110.

The coupling 116 may be formed integrally with the body portion 110 or the body portion 110 and the coupling 116 may be formed independently of one another and connected together thereafter by any suitable means.

The coupling 116 may comprise a bracket or clip 120. The clip 120 may include first and second arms, 122 and 124 respectively, each arm extending outwardly from a length of the body portion 110. In one embodiment, the arms 122 and 124 may substantially span the length of the coupling 116 and/or the body portion 110.

An enclosure 126 may be defined between the first and second arms 122 and 124 and may be arranged to receive a motor housing 128. In one embodiment, the first and second arms 122 and 124 extend towards each other to thereby define the enclosure 126, which may be configured as a substantially cylindrical or semi-cylindrical enclosure 126. In one embodiment, distal ends 154 and 156 of the arms 122 and 124, respectively, extend away from one another to thereby define a mouth 152 of the enclosure 126, wherein the mouth 152 facilitates insertion of the motor housing 128 into the enclosure 126.

The enclosure 126 may have a diameter that is substantially smaller than that of the motor housing 128, or may be sized substantially smaller than the motor housing 128, and the motor housing 128 may be inserted into the enclosure by pressing the motor housing 128 against the mouth 152 of the enclosure 126 to urge the arms 122, 124 away from one another to allow the motor housing 128 to be received and retained within the enclosure 126.

In another embodiment, the coupling 116 may comprise a retaining means such as a latch or a gate (not shown) arranged to extend from one distal end 154 to the other 156 to thereby retain the motor housing 128 securely in place within the enclosure 126. In yet another embodiment, the motor housing 128 may include a fastening means (not shown) for engaging with a fastening means (not shown) on the coupling 116 to enable the motor housing 128 to be securely retained within the enclosure 126.

In one embodiment, an internal surface or one or both of the first and second arms 122, 124 may include a keying feature, such as a marking or a tab 130, which is arranged to indicate a recommended positioning of the motor housing 128 within the enclosure 126. The motor housing 128 may include a corresponding marking or tab 132 for alignment with the tab 130 to facilitate the positioning of the motor housing 128 within the enclosure 126.

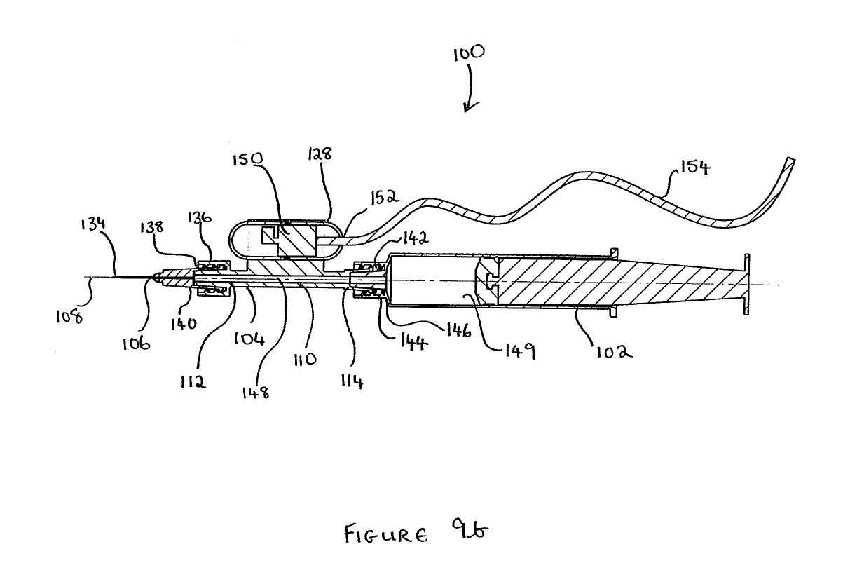

As more clearly depicted in FIGS. 9a and 9b, the first end portion 112 of the connector 104 may be connected to the medical instrument tip 106 arranged to receive a needle 108 at its distal end 134.

In one embodiment, the first end portion 112 of the connector 104 may comprise a male mating fitting 136 and may be arranged to engage with a female mating fitting 138 provided at a proximal end 140 of the medical instrument tip 106. In another embodiment, the first end portion 112 of the connector 104 may comprise a female mating fitting and may be arranged to engage with a male mating fitting provided at the proximal end 140 of the medical instrument tip 106.

The second end portion 114 of the connector 104 may be connected to the medical instrument body 102. In one embodiment, the second end portion 114 of connector 104 may comprise a female mating fitting 142 and may be arranged to engage with a male mating fitting 144 provided at a distal end 146 of the medical instrument body 102. In another embodiment, the second end portion 114 of the connector 104 may comprise a male mating fitting and may be arranged to engage with a female mating fitting provided at the distal end 146 of the medical instrument body 102.

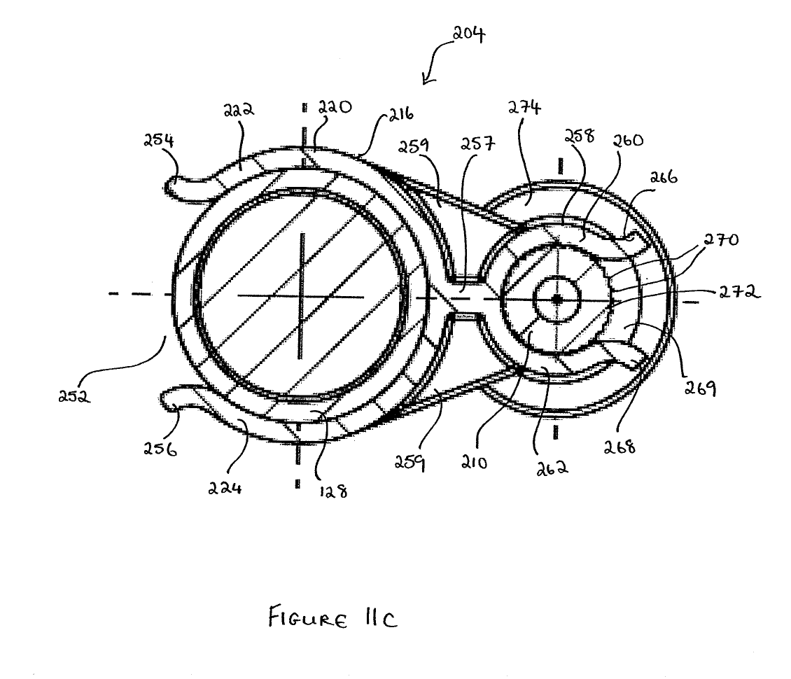

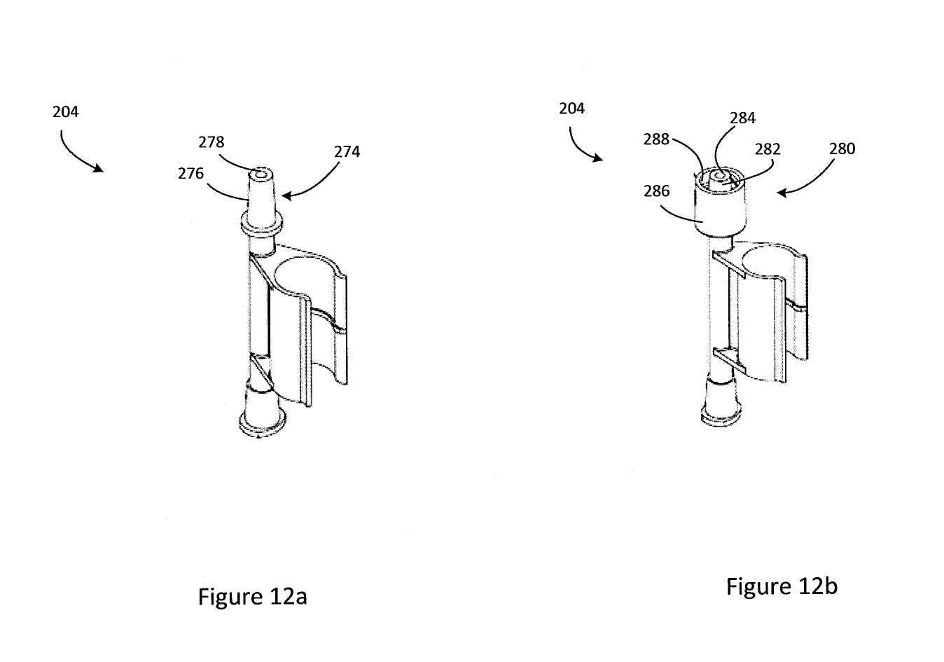

In some embodiments, the first end portion 112 and second end portion 114 of the connector 104 may be connected to the medical instrument tip 106 and the medical instrument body 102, respectively, by means of a Luer taper, such as a Luer lock or slip tip. A Luer taper is a standardised system of small-scale fluid fittings used for providing substantially leak-free connections between a male taper fitting and a corresponding female taper fitting of medical and laboratory instruments, and its key features are defined in the ISO 594 standards. A Luer taper may comprise a Luer-Lok® or a Luer-Slip®, both of which are discussed in more detail below with reference to FIGS. 12a and 12b. Advantageously, the connector 104 may be interconnected between standard medical instrument tips 106 and medical instrument bodies 102, without requiring any modification to the medical instrument tips 106 and medical instrument bodies 102.

The medical instrument body 102 may include an internal channel 148 extending along the length of the body portion 110, and in fluid communication with the first and second ends 112, 114 and accordingly with the needle 108 and an internal chamber 149 of the medical instrument body.

The motor housing 128 is arranged to receive a vibration motor 150. In one embodiment, the motor housing 128 includes an opening 152 to allow a cable 154 connected to the vibration motor 150 to exit the motor housing 128 for connection to an external power source and/or control mechanism (not shown).

Since the connector 104, and thus the coupling 116 retaining the motor housing 128 are securely connected to the medical instrument body 102 and medical instrument tip 106, the motor housing 128 is held in a fixed position and prevented from rotating about the medical instrument body 102 or tip 106 as a result of applied vibrations from the vibrating motor 150. In one embodiment, the connector 104 is effective to rigidly hold the vibrating motor 150 in a fixed position with respect to a bevel of the needle 108.

Furthermore, by interconnecting the connector 104 between the medical instrument body 102 and the medical instrument tip 106, the vibrating motor 150 is located in close proximity to the needle 108, and thereby provides a very effective transmission of vibration energy to the needle 108.

In one embodiment, the connector 104 is arranged to interconnect with the medical instrument body 102 and the medical instrument tip 106, such that when the connector 104 is secured to the body 102 and tip 106, the motor housing 128, and thus the vibrating motor 150, is positioned at a location substantially transverse to a cutting edge or piercing tip or bevel of the needle 106. Thus, when activated, the vibrating motor applies vibrations laterally to the body portion 110, and axis of movement of the medical instrument body 102 and tip 106, and so approximately or substantially at 90 degrees to the bevel of the needle 108, to thereby provide a shaper cutting edge or piercing tip.

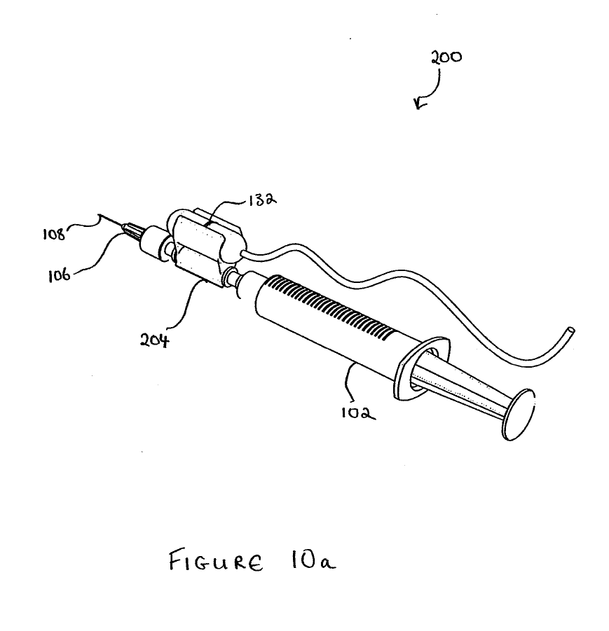

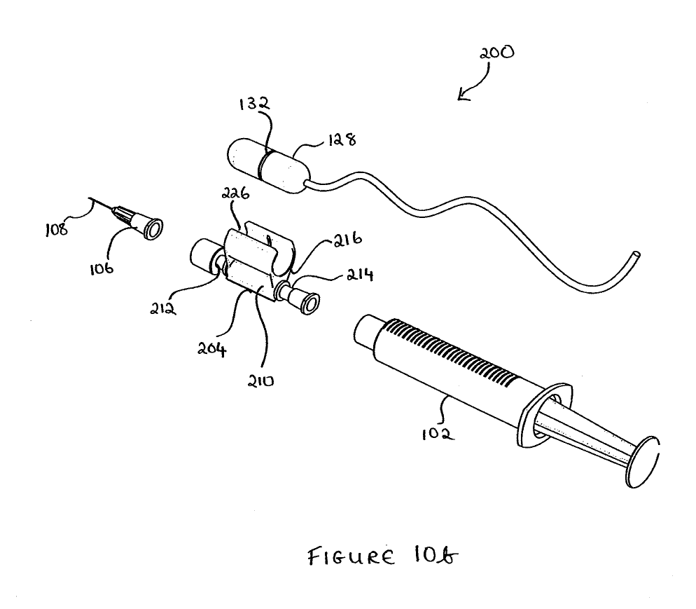

Referring now to FIGS. 10a and 10b, there is depicted a medical device 200 according to another embodiment. The medical device 220 comprises a similar medical instrument body 102, medical instrument tip 106, and needle 108 as those depicted in FIGS. 8a, 8b, 9a and 9b. However, instead of connector 104, medical device 220 includes a connector 204.

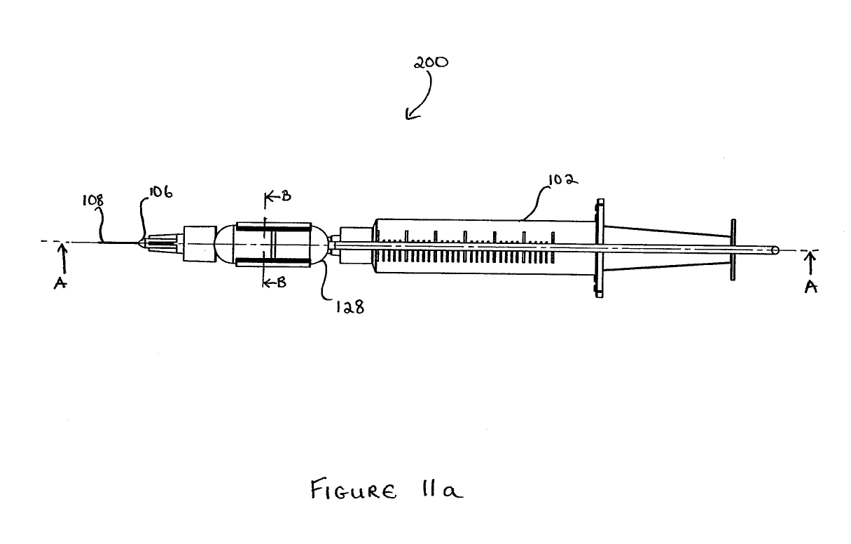

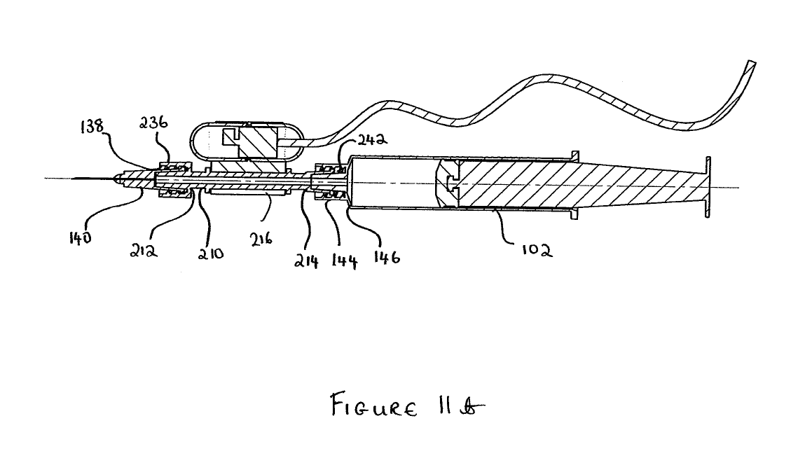

As illustrated in FIGS. 11a and 11b, the connector 204 may comprise a body portion 210 interconnecting a first and second end portion, 212, and 214, respectively. As with connector 104, the first end portion 212 of the connector 204 may comprise a male mating fitting 236 and may be arranged to engage with the female mating fitting 138 provided at the proximal end 140 of the medical instrument tip 106. In another embodiment, the first end portion 212 of the connector 204 may comprise a female mating fitting and may be arranged to engage with a male mating fitting provided at the proximal end 140 of the medical instrument tip 106.

Similarly, as with connector 104, the second end portion 214 of connector 204 may comprise a female mating fitting 242 and may be arranged to engage with a male mating fitting 144 provided at the distal end 146 of the medical instrument body 102. In another embodiment, the second end portion 214 of the connector 204 may comprise a male mating fitting and may be arranged to engage with a female mating fitting provided at the distal end 146 of the medical instrument body 102.

In some embodiments, the first end portion 212 and second end portion 214 of the connector 204 may be connected to the medical instrument tip 106 and the medical instrument body 102, respectively, by means of a Luer taper, such as a Luer-Lok® or a Luer-Slip®. A Luer taper may be composed of any suitable material, for example, metal or plastics.

Referring to FIG. 12a, there is depicted an embodiment wherein the second end portion 214 of connector 204 comprises a male fitting slip tip 274, such as a Luer-Slip®. The male fitting slip tip 274 comprises a substantially cylindrical member 276, which tapers towards its end, and includes a central bore 278. A female fitting slip tip (not shown), such as a Luer-Slip®, which may be provided on the medical instrument body 102 or the medical instrument tip 106, for example, comprises a substantially cylindrical member (not shown) and is arranged to receive the male fitting slip tip 274. In particular, the male fitting slip tip 274 is arranged to frictionally engage with the corresponding female fitting slip tip (not shown).

Referring to FIG. 12b, there is depicted an embodiment wherein the second end portion 214 of connector 204 comprises a male fitting Luer lock 278, such as a Luer-Lok®. The male fitting Luer lock 280 comprises a substantially cylindrical member 282, which tapers towards its end, and includes a central bore 284. The male fitting Luer lock 280 further comprises a sleeve portion 286 coaxial with the cylindrical member 282 and having an internal thread 288 disposed thereon. A female fitting Luer lock (not shown), such as Luer-Lok®, comprises a substantially cylindrical member (not shown) with external threads (not shown) disposed thereon. The female fitting Luer lock (not shown) is arranged to receive cylindrical member 282 of the male fitting Luer lock 280, and the internal thread 288 of the cylindrical member 282 is arranged to engage with the external threads of the cylindrical member (not shown) of the female fitting Luer lock. The cylindrical member 280 of the male fitting Luer lock 278 may also frictionally engage with the cylindrical member (not shown) of female fitting Luer lock (not shown).

Although FIGS. 12a and 12b depict a male fitting Luer lock and a male fitting slip tip respectively, disposed on the second end portion 214 of the connector 204, in some embodiments, the first end portion 212 and second end portion 214 of connector 204 and the first end portion 112 and second end portion 114 of connector 104 may comprise any of a male or female fitting Luer lock and slip tip. Advantageously, the connector 204 may be interconnected between standard medical instrument tips 106 and medical instrument bodies 102, without requiring any modification to the medical instrument tips 106 and medical instrument bodies 102.

Referring to FIGS. 10a, 10b, 11a, and 11b, the body portion 210 of the connector 204 may be a substantially longitudinal body portion and may be substantially cylindrical in shape. However, the body portion 210 may be shaped in any suitable manner, for example, such as a rectangular or hexagonal prism.

The connector 204 may further comprise a coupling 216. As most clearly depicted in FIG. 11c, the coupling 216 comprises a first bracket or clip 220 which is similar to the clip 120 of the connector 104 depicted in FIGS. 9a, 9b, 10a, and 10b. However, the clip 220 may include a spine portion 257, which extends along a length of the body portion 210.

The clip 220 may include first and second arms 222 and 224 respectively, each arm extending outwardly from the spine portion 257 and defining an enclosure 226 for receiving the motor housing 128. In one embodiment each arm 222 and 224 extends towards the other to thereby define a substantially cylindrically or semi-cylindrically shaped enclosure 226. In one embodiment, distal ends 254 and 256 of the arms 222 and 224, respectively, extend away from one another to thereby define a mouth 252 of the enclosure 226, wherein the mouth 252 facilitates insertion of the motor housing 128 into the enclosure 226.

The connector 204 may further comprise a second bracket or clip 258 including first and second arms 260 and 262 respectively, each arm extending outwardly from the spine portion 257 in a direction opposite or substantially opposite to a direction of the arms 222 and 224 of the first clip 220. In one embodiment, as depicted in FIG. 11c, a web 259 is provided to interconnect the first clip 220, the spine portion 257 and second clip 258.

In one embodiment, the first and second arms 260, 262 may extend toward one another to define therebetween a substantially cylindrically or semi-cylindrically shaped enclosure 264. The enclosure 264 is arranged to receive the body portion 210 to thereby connect or secure the body portion 210 of the connector 204 to the coupling 216. In one embodiment, distal ends 266 and 268 of the arms 260 and 262, respectively, extend away from one another to thereby define a mouth 269 of the enclosure 264

The enclosure 264 may have a diameter that is similar to or smaller than that of the body portion 210, or may be sized similar to or smaller than the body portion 210. Thus, pressing the body portion 210 against the mouth 269 of the enclosure 264 may urge the first and second arms 260 and 262 away from one another, allowing the body portion to be inserted into the enclosure 264 and to subsequently clasp the body portion 210.

The second clip 258, and accordingly the coupling 216, may be arranged to rotate about the body portion 210. In one embodiment, at least one detent 270 is provided on a surface 272 of the body portion 210 and may be provided to mechanically resist or arrest the rotation of the coupling 216 with respect to the body portion 210.

As depicted in FIG. 11c, a plurality of detents 270 may be disposed on a surface 272 of the body portion 210 and may extend substantially or partially around a circumference of the body portion 210. The detents 270 may be provided to divide a rotation of the coupling 216 with respect to the body portion 210 into a plurality of discrete increments. The detents 270 may engage with an internal surface of the clip 258 to hold the coupling 216 in a selected position with respect to the body portion 210. In one embodiment, the engagement between the detents 270 and the internal surface of the clip 258 is sufficient to substantially lock the coupling 216 to the body portion 210 in the selected position such that the coupling 216 and body portion 210 are restrained or substantially restrained from moving relative to one another when the vibrating motor is activated.

In one embodiment, a plurality of complimentary protrusions (not shown) is provided along an inner surface of the clip 258 and are arranged to engage with the detents 270. Thus, by availing of the plurality of detents 270 provided, the coupling 216 may be selectably adjusted to assume one of a plurality of states or positions with respect to the body portion 210.

In one embodiment, a collar 274 may be provided to encircle or enclose the second clip 258 and engage with the web 259. The first clip 220, the second clip 258, the spine 257 and web 259 may be integrally formed or may be formed independently and joined together thereafter. The body portion 210 and coupling 216 may be integrally formed or may be formed independently of one another and assembled thereafter.

Since the connector 204, and thus the coupling 216 retaining the motor housing 128 are securely connected to the medical instrument body 102 and medical instrument tip 106, the motor housing 128 is held in a fixed position and prevented from rotating about the medical instrument body 102 or tip 106 as a result of applied vibrations from the vibrating motor 150. In one embodiment, the connector 204 is effective to rigidly hold the vibrating motor 150 in a fixed position with respect to a bevel of the needle 108.

Furthermore, by interconnecting the connector 204 between the medical instrument body 102 and the medical instrument tip 106, the vibrating motor 150 is located in close proximity to the needle, and thereby provides a very effective transmission of vibration energy to the needle.

In one embodiment, the connector 204 is arranged to interconnect with the medical instrument body 102 and the medical instrument tip 106, such that when the connector 204 is secured to the body 102 and tip 106, the motor housing, and thus the vibrating motor, is positioned at a location substantially transverse to a cutting edge or piercing tip or bevel of the needle 106.

However, connector 204 further allows a user to adjust or modify the position or location of the coupling 216 with respect to the body portion 210 by rotating the coupling 216 about the body portion 210 in increments to better position the vibrating motor 150 and provide for a more effective transmission of the vibrations to the needle 108 as required.

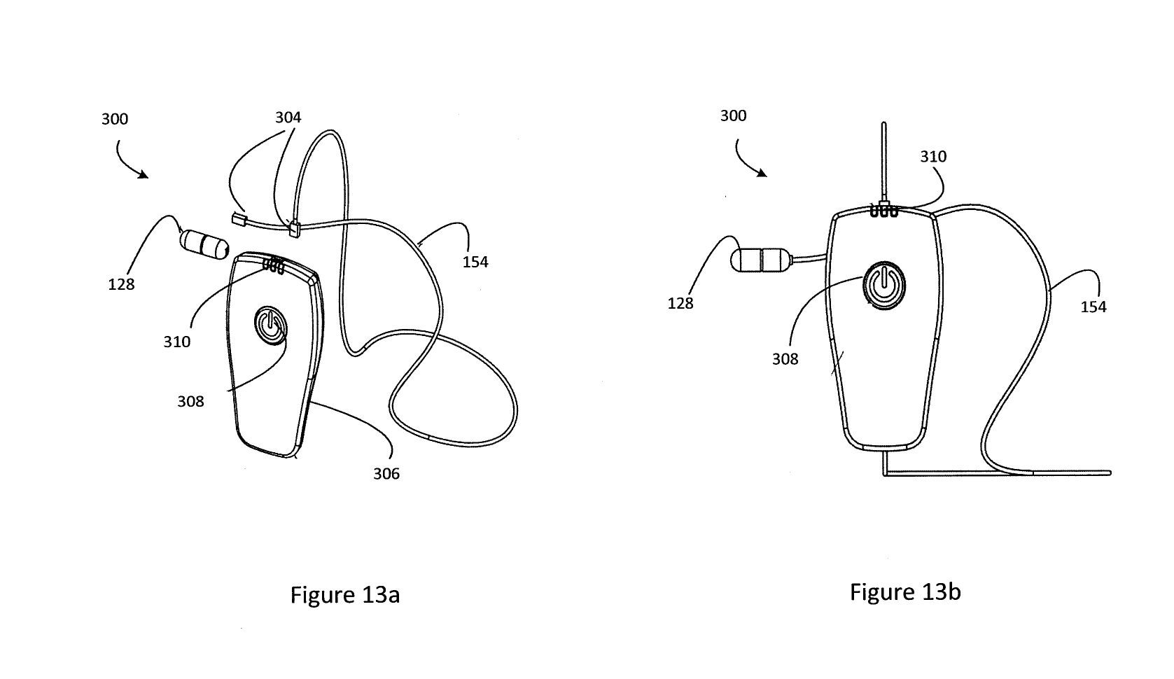

Referring now to FIGS. 13a and 13b, there is depicted a control system 300. In some embodiments, the control system 300 comprises a controller 302 arranged to electrically connect to the vibrating motor 150 provided within the motor housing 128 by means of the cable 154. In some embodiments, the cable 154 comprises detachable plugs 304 provided at either end of the cable 154 and arranged to detachably engage with or connect to ports (not shown) provided on the controller 302 and vibrating motor and/or motor housing 128, respectively.

In some embodiments, the controller 302 is a battery powered controller and may comprise a compartment 306 having battery terminals (not shown) for electrically connecting at least one battery (not shown) to the controller 302. The battery may be replaceable and/or rechargeable. The controller 302 may be a hand held controller. In some embodiments, the controller 302 may be arranged to connect to a user or physician, for example, with an attachment means, such as a clip or strap, which may engage with a belt, loop, fastener or the like, provided on the user.

As depicted, the controller 302 may further include an on/off or activation/deactivation control 308 and may include a display 310 for indicating whether the controller 302 is activated or deactivated. In some embodiments, the display 310 comprises at least one LED.

In some embodiments, when the controller 302 is connected to the motor housing 128, and in particular, the vibrating motor 150 provided within the motor housing 128, activation of the activation/deactivation control 308 causes power to be transmitted from the controller 302 through the cable 154 to the vibrating device 150 to cause the vibrating device 150 to vibrate at a given frequency. Thus, in some embodiments, the frequency at which the vibrating motor 150 vibrates is a function of the power transmitted to the vibrating motor 150 from the controller 302. In some embodiments, the controller 302 includes a user interface or dial (not shown) for adjusting the power being transmitted by the controller 302 and accordingly, the frequency at which the vibrating device 150 vibrates or is to vibrate.

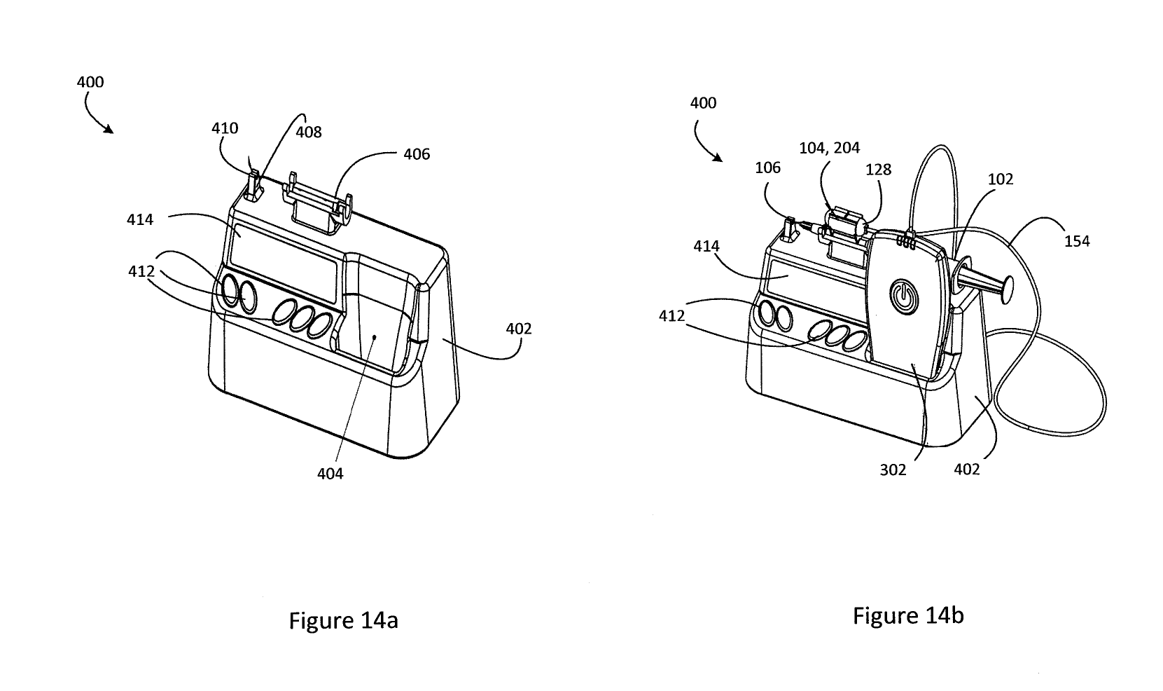

Referring to FIGS. 14a and 14b, there is depicted a control system dock 400. The control system dock 400 comprises a base unit 402 having a controller cradle 404 arranged to receive the controller 302. In some embodiments, the control system dock 400 is arranged to connect to a power supply, such as a mains power supply. In some embodiments, the control system dock is arranged to receive a 240V AC input.

The controller cradle 404 may be arranged to transmit power to the controller 302 whilst the controller is docked in the controller cradle 404, to thereby recharge rechargeable batteries provided in the compartment 306 of the controller 302.

In some embodiments, the control system dock 400 includes a holder or fixture 406 for receiving the medical device 100, 200 and a sensor 408, for example, a frequency or motion sensor, disposed in proximity to the fixture 406 such that when the medical device 100, 200 is located on the fixture, the medical instrument tip 106 is in close proximity to the sensor such that movements of medical instrument tip 106 are capable of being sensed by the sensor 408. In some embodiments, the medical instrument tip 106 is arranged to extend within a gap 410 defined by the sensor 408.

In some embodiments, the control system dock 400 comprises a processor (not shown) which is arranged to receive readings from the sensor 408 for processing. The control system dock 400 may further comprise a user interface, such as a set of user actuatable controls, 412, by which user inputs may be received by the processor (not shown). The control system dock 400 may further comprise a display 414 for displaying readings and/or selected or actuated controls 412.

In some embodiments, the control system dock 400 is employed to program the controller 302 to operate in accordance with a set of operating instructions. For example, the processor (not shown) of the control system dock 400 may transmit control instructions to the controller 302 via WiFi, Bluetooth and/or a physical connection, to program the controller 302 to operate in accordance with the set of operating instructions. In some embodiments, the operating instructions comprise at least a power setting for transmitting power to the vibrating device 150 to cause the vibrating device to vibrate at a particular frequency. In some embodiments, the user interface or dial (not shown) of the controller 302 may be employed to adjust the power being transmitted by the controller 302, and thus the vibration frequency, for example, to override the operating instructions. In some embodiments, adjustment of the power being transmitted to the controller 302 via user interface may cause a signal, such as a feedback signal, to be transmitted to the processor of the control system dock 400 indicative of the adjustment or updated value of power being transmitted. In some embodiments, the processor 302 may store the adjustment or updated value in a memory and/or may utilise the adjustment or updated value in future calibrations of the controller and/or tuning of the medical device.

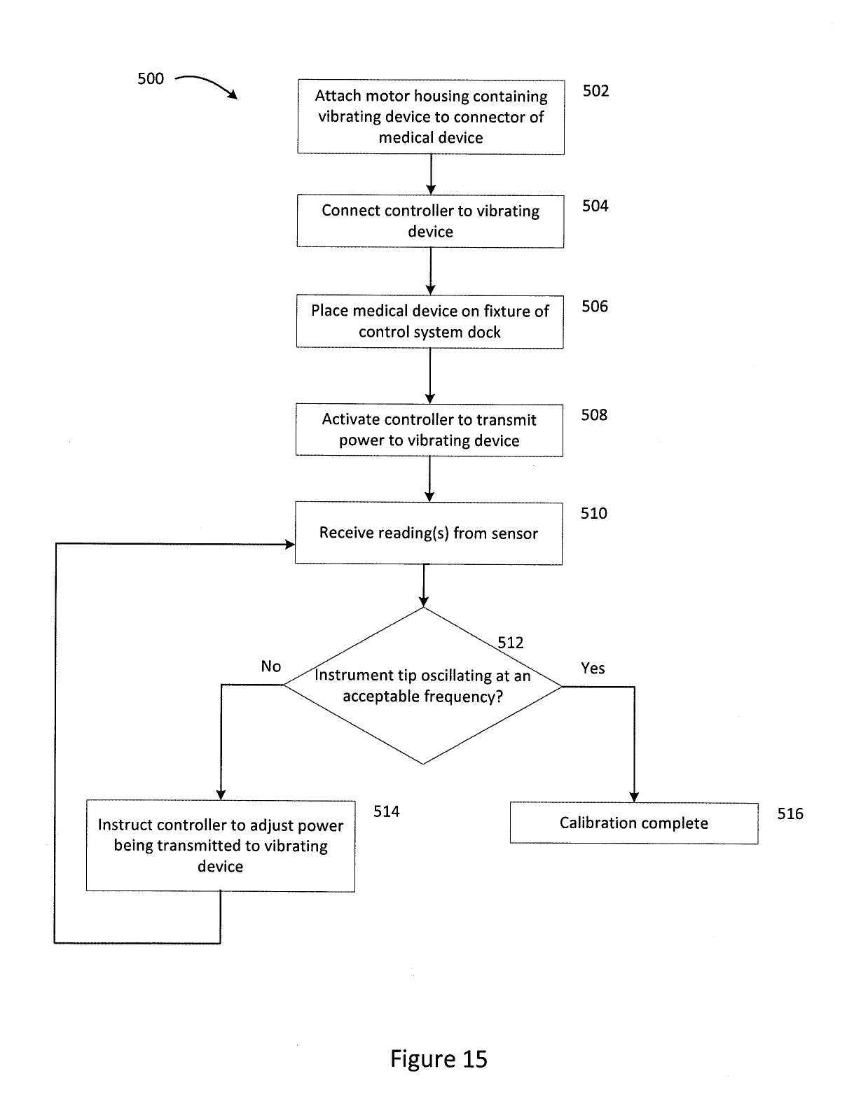

In some embodiments, the control system dock 400 is employed to calibrate or tune the controller 302 with respect to the medical device 100, 200, as described with reference to FIG. 15. FIG. 15 is a flow diagram depicting a method 500 of calibrating the controller 302 to cooperate with the vibrating device to cause the medical instrument tip 106 to oscillate at a threshold or optimum frequency, according to some embodiments.

At 502, the medical device 100, 200 is assembled by attaching the motor housing 128 including the vibrating device 150 to the instrument body 102 and instrument tip 106 via connector 104, 204. At 504, the controller 302 is connected to the vibrating device 150, for example, by means of the cable 154. At 506, the medical device 100, 200, is placed on the fixture 406 of the control system dock 400 such that the instrument tip 106 is positioned in proximity to the sensor 408, and in some embodiments, such that the instrument tip 106 extends within the gap 410 defined by the sensor 408.

At 508, the controller 308 is activated, for example, by a user activating the activation/deactivation control 308 provided on the controller 302 and power is transmitted to the vibrating device 150 in accordance with a power setting of the controller 302. As a result, the vibrating device 150, and thus the instrument tip 106 to which the vibrating motor 150 is attached, is caused to vibrate or oscillate at a frequency that is a function of the power received from the controller 302.

At 510, the sensor 408 senses the vibration or oscillation frequency of the instrument tip 106 and provides a reading of the sensed frequency to the processor of the control system dock 400. In some embodiments, the sensor 408 senses the oscillation frequency and amplitude of the instrument tip 106 and the reading comprises the sensed frequency and the sensed amplitude values. At 512, the processor compares the reading with an acceptable range, such as a threshold value or range, for example, an optimum frequency value, and which may be a pre-configured value or range stored in a memory (not shown) associated with the processor. In some embodiments, the processor may cause the readings to be display on display 414.

At 514, if the reading is not deemed acceptable for example, if it does not correspond or substantially correspond with the threshold value or is not within the threshold range, the processor transmits a signal to the controller 302 to cause the control to adjust the amount of power being transmitted to the vibrating device 150. For example, the processor may transmit a signal to instruct or program the controller 302 to increase or to decrease the amount power being transmitted to the vibrating device 150. In some embodiments, the reading is displayed on the display 414 and the processor transmits the signal to the controller 302 in response to user inputs received via the user actuatable controls, 412.

The method then reverts to 510 and the sensor 408 senses the vibration or oscillation frequency of the instrument tip 106 and provides a reading of the sensed frequency to the processor of the control system dock 400. Again, in some embodiments, the sensor 408 senses the oscillation frequency and amplitude of the instrument tip 106 and the reading comprises the sensed frequency and the sensed amplitude values. At 512, the processor compares the reading with an acceptable range, such as a threshold frequency value or range, for example, an optimum frequency value. Thus, steps 510 to 514 are repeated until the reading corresponds with or substantially corresponds with an acceptable range, such as a threshold value or threshold range.

At 516, once the reading corresponds with or substantially corresponds with a threshold value or is within a threshold range, the calibration method is complete. In this way, the controller 302 may be programmed to transmit an amount of power to the vibrating device to cause the instrument tip 106 to operate at a threshold frequency, such as an optimum frequency or an optimum penetration frequency.

The word ‘comprising’ and forms of the word ‘comprising’ as used in this description and in the claims does not limit the invention claimed to exclude any variants or additions.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A connector for use with a medical device, the connector comprising:
a body portion having a first end and a second end;
the body portion including an internal channel extending along a length of the body portion providing fluid communication between the first end and the second end; and a coupling connected to the body portion;
the coupling comprising a first bracket arranged to receive a motor housing;
wherein the first end is arranged to connect to an instrument body of the medical device and the second end is arranged to connect to an instrument tip of the medical device.

2. The connector of claim 1, wherein the coupling is rigidly attached to the body portion.

3. The connector of claim 1, wherein the first bracket comprises first and second arms, each arm extending outwardly from the body portion to define an enclosure for receiving the motor housing.

4. The connector of claim 3 wherein each arm extends substantially toward one another.

5. The connector of claim 1, wherein the coupling further comprises a second bracket arranged to engage with the body portion.

6. The connector of claim 5, wherein the coupling comprises a spine portion, and wherein the first bracket extends from the spine in a first direction and the second bracket extends from the spine in a second direction, different from the first direction.

7. The connector of claim 6, wherein the second bracket comprises first and second arms, each arm extending outwardly from the spine of the coupling and substantially toward one another to define an enclosure for receiving the body portion.

8. The connector of claim 7, wherein a plurality of detente is disposed on a surface of the body portion and are arranged to engage with an inner surface of the second bracket to thereby allow the coupling to be rotated incrementally about the body portion.

9. The connector of claim 8, wherein the plurality of detente extend at least partially around a circumference of the body portion to thereby provide a plurality of discrete increments by which the coupling may be rotated incrementally about the body portion.

10. The connector of claim 1, wherein a fitting is provided at the first end of the body portion and is arranged to cooperate with a corresponding fitting provided on the instrument body.

11. The connector of claim 1, wherein a fitting is provided at the second end of the body portion and is arranged to cooperate with a corresponding fitting provided on the instrument tip.

12. The connector of claim 10, wherein the fitting comprises a Luer taper.

13. A medical device comprising an instrument body, an instrument tip including a needle and a connector according to claim 1, wherein the connector allows fluid communication between a chamber of the instrument body and the needle.

14. The medical device of claim 13, wherein the needle comprises a cutting edge and the motor housing includes a vibrator, wherein the vibrator is arranged to cause the cutting edge of the needle to vibrate.

15. The medical device of claim 14, wherein the vibrator is arranged to cause the cutting edge to vibrate at an optimum penetration frequency of the cutting edge.

16. The medical device according to claim 15, wherein the optimum penetration frequency is a frequency range at which there is substantial reduction in the peak penetration force when measured using a tensile testing instrument.

17. The medical device according to claim 14, wherein the cutting edge is moveable along an axis, and the vibrator is arranged to cause the cutting edge to vibrate in a motion substantially transverse to said axis.

18. The medical device according to claim 17, wherein the medical device is arranged to cause the cutting edge to vibrate in a circular motion substantially transverse to said axis.

19. The medical device according to claim 14, further comprising a controller for selectively causing said vibrator to vibrate at the optimum penetration frequency.

20. The medical device according to claim 19, wherein the controller for selectively causing the vibrator to vibrate at the optimum penetration frequency includes:
(a) a first sensor for sensing the ease by which the cutting edge penetrates body tissue;
(b) a second sensor for sensing the frequency of the vibration of the vibrator;
(c) a correlation device to correlate the measurements sensed by the first and second sensors and provide an optimised feedback signal; and
(d) an adjuster to select the frequency for the vibration of the vibrator in response to the feedback signal.

21. The medical device according to claim 14, wherein the coupling is adapted to attach to a range of medical instruments having differing sized bodies.

22. The medical device according to claim 14, wherein the instrument body has a longitudinal axis, and said cutting edge is remote from said body and moveable along the axis.

23. The medical device according to claim 13, wherein said medical device is selected from the group consisting of scalpers, lancets and syringes.

24. The medical device according to claim 13, wherein the medical device is a syringe capable of expressing a liquid, and wherein when the vibrator is arranged to cause the syringe to vibrate so that the liquid is expressed in a vortex.

25. The medical device according to claim 13, wherein said medical device is a syringe for use in a procedure selected from the group consisting of sclerotherapy procedures, facial injection procedures and dermal filter procedures.

26. The medical device according to claim 13, wherein the first bracket comprises a resilient spring or clip which minimises any motion of the motor housing relative to the instrument tip and enables the vibrations to be transmitted to the instrument tip, and wherein the spring or clip does not significantly compress or distort the instrument tip or body.

27. The medical device according to claim 26, wherein the connector comprises a spring having a first winding in a clockwise direction and a second winding in an anticlockwise direction.

28. (canceled)

29. A control system dock comprising:
a base unit including a processor;
a controller cradle arranged to receive a controller;
a fixture arranged to receive a medical device; and
a sensor;
wherein the processor is operable to:
receive signals from the sensor relating to a measure of vibration of an instrument tip of the medical device; and
transmit signals to the controller to program the controller to transmit power to a vibrating motor connected to the medical device.

30. The control system dock of claim 29, wherein the processor is operable to program the controller to transmit an amount of power to the vibrating motor to cause the vibrating motor to vibrate at a particular frequency.

31. The control system dock of claim 29, wherein processor is operable to communicate with the controller via any one of WiFi; Bluetooth, or a physical connector.

32. The control system dock of claim 29, wherein the controller cradle includes electrical terminals arranged to electrically connect to the controller and arranged to transmit power to the controller.

33. The control system dock of any of claim 29, wherein the sensor comprises at least one of frequency sensor and a motion sensor.

34. The control system dock of claim 29, wherein the processor is arranged to receive user inputs via user actuatable controls provided on the control system dock.

35. The control system dock of claim 29, wherein the processor is arranged to provide outputs via a display provided on the control system dock.

36. The control system dock of claim 29, wherein the processor is further operable to receive a feedback signal from the controller in response to an adjustment of the power being transmitted by the controller.

37. The control system dock of claim 36, wherein information derived from the feedback signal is employed for further operations.

38. The control system dock of claim 29, further comprising a controller provided in the controller cradle and a medical instrument provided on the fixture, wherein the medical device comprises a connector including a vibrating motor.

39. The control system dock of claim 38, wherein the controller comprises a user interface to allow for adjustment of the power to be transmitted to the vibrating motor.

40. (canceled)

41. A method of calibrating a controller for use with a medical device, the method comprising:
transmitting power from a controller to a vibrating device connected to a medical device;
receiving a measurement value indicative of a vibration of a medical tip of the medical device; and
subject to determining that the measurement value does not fall within an acceptable range, transmitting from a processor to the controller, a signal to adjust an amount of power being transmitted to the vibrating device.

42. The method of claim 41 further comprising at the controller, adjusting the amount of power being transmitted to the vibrating device.

43. The method of claim 42 wherein the adjusting comprises at least one of increasing or decreasing the amount of power being transmitted to the vibrating device.

44. The method of claim 41, wherein determining that the measurement value does not fall within an acceptable range comprises the processor comparing the measurement value with threshold value stored in a memory.

45. The method of claim 41, wherein determining that the measurement does not fall within an acceptable range comprises providing a user with an indication of the measurement and receiving a user input indicative of whether or not the measurement falls within an acceptable range.

46. The method of claim 41, further comprising transmitting the signal to adjust an amount of power being transmitted to the vibrating device in response to a user input.

47. The method of claim 41, wherein receiving a measurement value indicative of a vibration of a tip of the medical device comprises sensing at least one of an oscillation frequency of the tip and an oscillation amplitude of the tip.

48. The method of claim 41, further comprising receiving a feedback signal from the controller in response to an adjustment of the power being transmitted by the controller.

49. The method of claim 48, wherein the adjustment of the power being transmitted by the controller is in response to a user input received via a user interface provided on the controller.

50. The method of claim 48, wherein information derived from the feedback signal is employed for further operations.

51. A method of reducing the resistance to penetration of a body tissue by a cutting edge on a medical instrument, the penetration of said body tissue occurring along an axis, the method comprising causing the cutting edge to vibrate at the optimum penetration frequency of the cutting edge.

52. A method of reducing the static friction between a cutting edge and a body tissue when said cutting edge is forced against said body tissue along an axis, the method comprising causing the cutting edge to vibrate at the optimum penetration frequency of the cutting edge.

53. The method according to claim 51, wherein the method further comprises causing said cutting edge to vibrate in a motion substantially transverse to said axis.

54. The method according to claim 53, wherein said motion is a circular motion substantially transverse to said axis.

55. The method according to claim 51, wherein said cutting edge is the tip of a needle.

56. The method according to claim 51, for use in a procedure selected from the group consisting of sclerotherapy procedures, facial injection procedures and dermal filler procedures.
